(19) United States
(12) Patent Application Publication
(10) Pub. No.: US 2008/0154264 A1
(43) Pub. Date: Jun. 26, 2008

(54) Cannulated grooves to aid in cannula to guide quick connection

(75) Inventors: Michael A. Wack, Warsaw, IN (US); Daniel W. Buehler, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 11/644,130

(22) Filed: Dec. 22, 2006

(51) Int. Cl. A61B 17/72

(52) U.S. Cl. 606/64

(57) ABSTRACT

A screw cannula useable with an intramedullary nail system for use with an anatomical structure, such as a femur, for example. The screw cannula may include a groove formed in an exterior surface of the cannula. The groove provides a positive locking mechanism with audible and tactile feedback for a surgeon. The groove additionally provides infinite adjustability of the cannula relative to the anatomical structure.

### BACKGROUND

1. Field of the Invention

The present invention relates to an intramedullary nail system. More particularly, the present invention relates to a screw cannula useable with an intramedullary nail system.

2. Description of the Related Art

Intramedullary nail systems typically include an intramedullary nail, a targeting guide, and a screw cannula used to position the nail and associated fixation screws in an intramedullary canal of an anatomical structure, such as a femur.

### SUMMARY

The present disclosure provides a screw cannula useable with an intramedullary nail system for use with an anatomical structure, such as a femur, for example. The screw cannula may include a groove formed in an exterior surface of the cannula. The groove provides a positive locking mechanism with audible and tactile feedback for a surgeon. The groove additionally provides infinite adjustability of the cannula relative to the anatomical structure.

In one form thereof, the present disclosure provides a cannula for use with an intramedullary nail system including a targeting guide, the cannula including a hollow shaft, the shaft defining an external surface; and at least one groove, the groove formed in the external surface of the shaft, the groove configured to engage the targeting guide in a plurality of discrete locations.

In another form thereof, the present disclosure provides an intramedullary nail system for use with an anatomical structure, including a targeting guide; an intramedullary nail; and a cannula comprising locking means for positively locking the cannula relative to the targeting guide in a plurality of discrete locations.

In yet another form thereof, the present disclosure provides a method for using an intramedullary nail system including a targeting guide and an intramedullary nail, the intramedullary nail for implantation in an anatomical structure, the method including the steps of providing a cannula, the cannula including a hollow shaft, the shaft defining an external surface; and at least one groove, the groove formed in the external surface of the shaft, the groove configured to engage the targeting guide in a plurality of discrete locations; inserting the cannula into the targeting guide; and axially moving the cannula toward the anatomical structure until the groove engages the targeting guide.

### BRIEF DESCRIPTION OF THE DRAWINGS

The above-mentioned and other features of this disclosure, and the manner of attaining them, will become more apparent and will be better understood by reference to the following description of embodiments of the disclosure taken in conjunction with the accompanying drawings, wherein:

FIG. 1 is an exploded perspective view of an intramedullary nail system according to an exemplary embodiment of the present disclosure;

FIG. 2 is a fragmented perspective view of a portion of the intramedullary nail system of FIG. 1;

FIG. 3 is another fragmented perspective view of a portion of the intramedullary nail system of FIG. 1; and

FIG. 4 is yet another fragmented perspective view of a portion of the intramedullary nail system of FIG. 1.

Corresponding reference characters indicate corresponding parts throughout the several views. The exemplifications set out herein illustrate embodiments of the disclosure and such exemplifications are not to be construed as limiting the scope of the invention in any manner.

### DETAILED DESCRIPTION

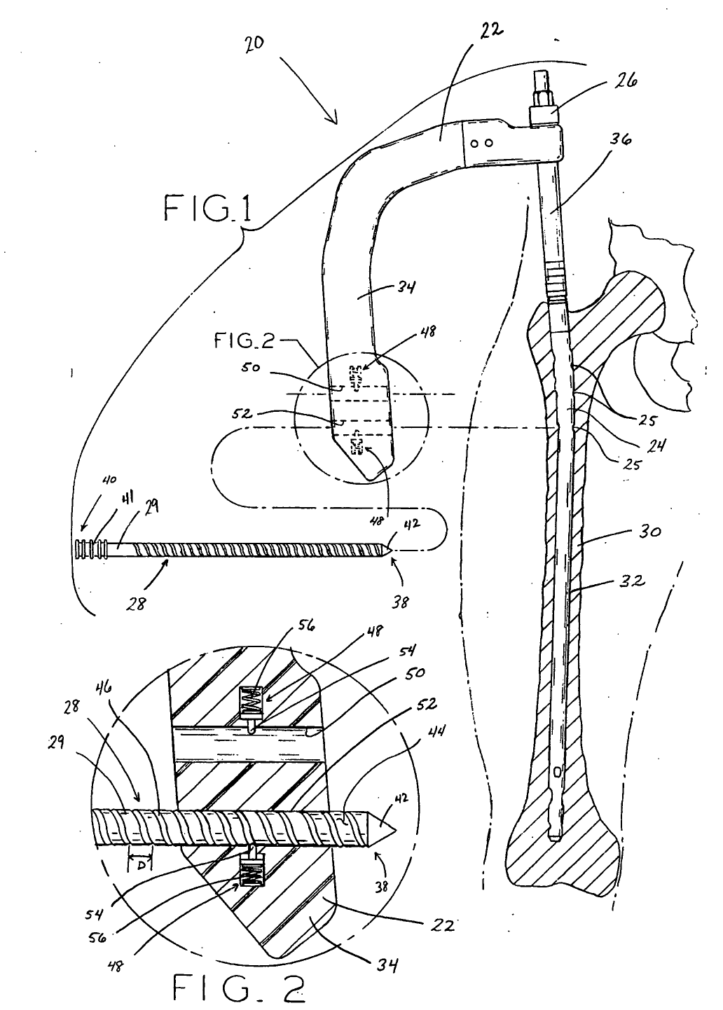

Referring to FIGS. 1 and 2, intramedullary (IM) nail system 20 is shown and generally may include targeting guide 22, IM nail 24, connection or locking bolt or screw 26, and cannula 28. IM nail system 20 is useable in an anatomical structure, such as femur 30, for example, having IM canal 32. Cannula 28 may be a screw cannula to provide an unobstructed portal for passage of a screw through soft tissue 31 and into femur 30 and screw bores 25 of IM nail 24. Cannula 28 may also provide an unobstructed portal for passage of other materials and/or tools through soft tissue 31 and into an anatomical structure. Targeting guide 22 may include alignment or arm portion 34 and connection portion 36. Arm portion 34 may include upper throughbore 50 and lower throughbore 52 for engagement with cannula 28, as described below.

Referring still to FIGS. 1 and 2, cannula 28 may include hollow shaft 29 which defines proximal end 40 and distal end 38. When used with reference to a surgical instrument, the term “distal” is meant to indicate a portion of the instrument farthest away from a user, such as a surgeon, and the term “proximal” is meant to indicate a portion of the instrument closest to the user. Distal end 38 may include trocar 42 for facilitating piercing of soft tissue 31 and stabilizing cannula 28 on femur 30, as described below. Proximal end 40 may include grip portion 41 for facilitating ease of use of cannula 28 for a surgeon. Cannula 28 includes exterior surface 44 having a substantially constant cross-sectional diameter from proximal end 40 to distal end 38. Cannula 28 may also include groove or recess 46 formed in exterior surface 44. In an exemplary embodiment, groove 46 is a spiral groove which extends continuously along a substantial length of cannula 28 from proximal end 40 to distal end 38. In one embodiment, groove 46 has a general hemispherical cross-sectional shape which defines a groove radius. Groove 46 may be shaped with a substantially identical shape and radius as that of detent 54, as described below.

Each throughbore 50, 52 of targeting guide 22 may include detent mechanism 48 having detent 54 and spring 56. In one embodiment, detent 54 has a general hemispherical cross-sectional shape and defines a ball radius matching that of the groove radius of groove 46. Detent 54 is shown positioned in groove 46 in FIG. 2, for example.

In operation and referring to FIGS. 1-4, cannula 28 may be used in a method for implanting IM nail 24 in femur 30. As shown in FIG. 1, targeting guide 22 and IM nail 24 are assembled together with connection bolt 26. IM nail 24 is then implanted into femur 30. Targeting guide 22 provides throughbores 50, 52, for example, which facilitate alignment of IM nail screws into screw bores 25 of IM nail 24. Cannula 28 may be inserted into either throughbore 50 or 52. Alternatively, a plurality of cannulas 28 may be provided and inserted into both throughbores 50 and 52 simultaneously. In an exemplary embodiment, cannula 28 is inserted into throughbore 50 after which a screw is inserted into a corresponding screw bore 25 and then cannula 28 is removed from throughbore 50 and inserted into throughbore 52 after which a screw is inserted into the corresponding screw bore 25, or vice versa.

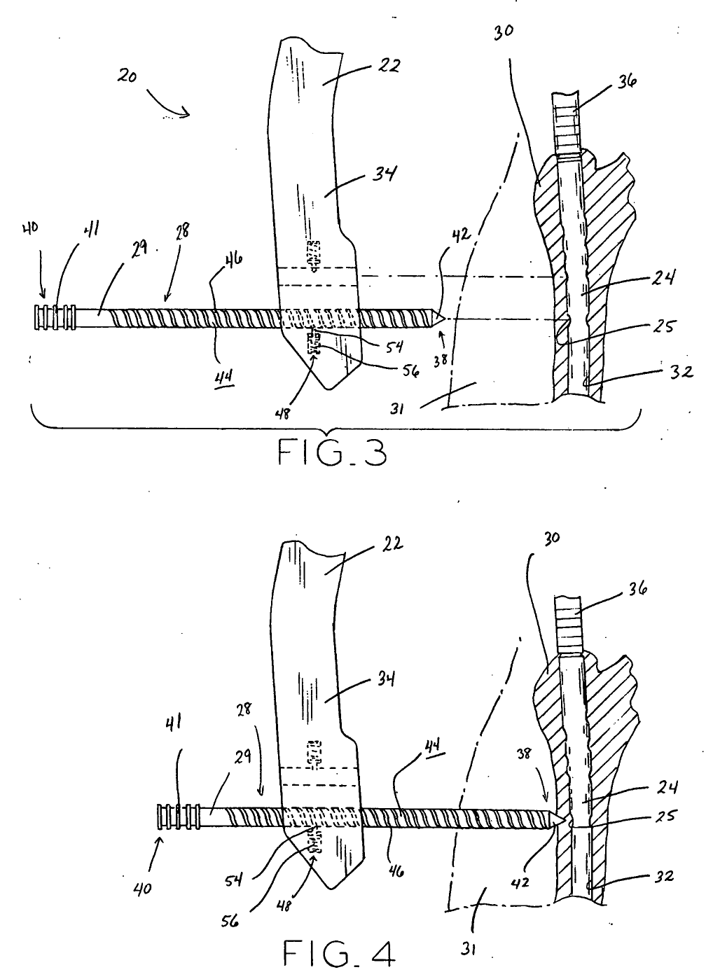

As shown in FIGS. 2 and 3, cannula 28 is inserted into throughbore 52 by passing trocar 42 and distal end 38 of cannula 28 therethrough until trocar 42 almost touches soft tissue 31. Once cannula 28 is sufficiently inserted into throughbore 52, detent 54 engages exterior surface 44 of cannula 28. Spring 56 of detent mechanism 48 maintains a constant pressure between detent 54 and exterior surface 44, i.e., detent 54 presses against spring 56 to create compression between detent 54 and exterior surface 44. Movement of cannula 28 further toward soft tissue 31 results in detent 54 encountering groove 46. Upon encountering groove 46, the force from spring 56 forces detent 54 to engage groove 46. Spring 56 maintains a pressure between detent 54 and cannula 28 when detent 54 is engaged with groove 46.

Upon further movement of cannula 28 toward soft tissue 31, detent 54 reengages exterior surface 44 of cannula 28 until detent 54 again encounters groove 46. Each time that detent 54 encounters groove 46, a clicking or otherwise audible sound is produced which indicates to the surgeon that cannula 28 is positively locked to targeting guide 22. In an exemplary embodiment, detent 54 and groove 46 have substantially identical cross-sectional shapes and/or radii to facilitate a secure connection between targeting guide 22 and cannula 28. In this manner, the engagement of detent 54 with groove 46 maintains a secure connection between targeting guide 22 and cannula 28 to prevent cannula 28 from unintentional disengagement with targeting guide 22 during a surgical procedure. Furthermore, engagement of detent 54 with groove 46 provides a positive tactile feedback to the surgeon that cannula 28 is locked to targeting guide 22. Advantageously, such tactile and audible confirmation of a locking engagement allows the surgeon to leave cannula 28 unattended in targeting guide 22 while performing other parts of the surgical procedure. In one embodiment, a visual confirmation may provide the positive feedback to the surgeon.

As shown in FIGS. 2 and 4, upon further movement of cannula 28 toward femur 30, trocar 42 pierces soft tissue 31 and cannula 28 enters soft tissue 31. Once inserted through soft tissue 31, trocar 42 abuts femur 30 to facilitate locating and stabilizing cannula 28 on femur 30. Cannula 28 thus provides a cannulated portal to permit unobstructed passage of a screw, materials, or other tools through soft tissue 31. In an exemplary embodiment, cannula 28 provides access for a screw and associated driver to be inserted through soft tissue 31 for engaging a screw bore 25.

If a surgeon encounters a situation in which, during movement of cannula 28 toward femur 30, the surgeon needs to move cannula 28 a distance shorter than distance D (FIG. 2), either toward femur 30 or away from femur 30, the surgeon may simply first ensure that detent 54 is engaged with groove 46 via audible, tactile, and/or visual confirmation, and then rotate cannula 28 in an appropriate direction, such as clockwise for movement toward femur 30 and counterclockwise for movement away from femur 30, for example. Detent 54 follows a path defined by groove 46 around cannula 28 during rotation of cannula 28. Engagement of detent 54 with groove 46 ensures that cannula 28 remains positively locked with targeting guide 22 during rotation of cannula 28 and advantageously allows an infinite adjustability of cannula 28 relative to femur 30.

In an alternative embodiment, cannula 28 includes a plurality of spaced grooves progressively positioned along the length of cannula 28. Such plurality of spaced grooves may provide the audible, tactile, and/or visual feedback, as described above, upon insertion of cannula 28 into targeting guide 22.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A cannula for use with an intramedullary nail system including a targeting guide, the cannula comprising:
a hollow shaft, said shaft defining an external surface; and
at least one groove, said groove formed in said external surface of said shaft, said groove configured to engage the targeting guide in a plurality of discrete locations.

2. The cannula of claim 1, wherein said groove is configured to engage the targeting guide in an infinitely adjustable manner.

3. The cannula of claim 1, wherein said groove comprises a substantially continuous groove.

4. The cannula of claim 1, wherein said groove comprises a spiral groove.

5. The cannula of claim 1, wherein the targeting guide comprises a spring-actuated engagement structure, said spring-actuated engagement structure sized for receipt in said groove when in one of said plurality of discrete locations.

6. The cannula of claim 1, wherein said groove is configured to provide an audible indication of a positive locking engagement between the targeting guide and the cannula.

7. The cannula of claim 1, wherein said groove is configured to provide a tactile indication of a positive locking engagement between the targeting guide and the cannula.

8. An intramedullary nail system for use with an anatomical structure, comprising:
a targeting guide;
an intramedullary nail; and
a cannula comprising locking means for positively locking the cannula relative to the targeting guide in a plurality of discrete locations.

9. The intramedullary nail system of claim 8, wherein said cannula further comprises adjustment means for permitting infinite adjustment of the cannula relative to the anatomical structure.

10. The intramedullary nail system of claim 8, wherein said locking means comprises an audible indication means.

11. The intramedullary nail system of claim 8, wherein said locking means comprises a tactile indication means.

12. A method for using an intramedullary nail system including a targeting guide and an intramedullary nail, the intramedullary nail for implantation in an anatomical structure, the method comprising the steps of:
providing a cannula, the cannula comprising:
a hollow shaft, the shaft defining an external surface; and
at least one groove, the groove formed in the external surface of the shaft, the groove configured to engage the targeting guide in a plurality of discrete locations;
inserting the cannula into the targeting guide; and
axially moving the cannula toward the anatomical structure until the groove engages the targeting guide.

13. The method of claim 12, further comprising the step of rotating the cannula relative to the targeting guide subsequent to said moving step, said rotating step axially moving the cannula relative to the anatomical structure in an infinitely adjustable manner.

14. The method of claim 12, further comprising the steps of axially moving the cannula toward the anatomical structure such that the groove engages the targeting guide more than once, wherein upon each engagement of the targeting guide and the groove, an audible indication of a positive locking engagement is provided.

15. The method of claim 12, further comprising the steps of axially moving the cannula toward the anatomical structure such that the groove engages the targeting guide more than once, wherein upon each engagement of the targeting guide and the groove, a tactile indication of a positive locking engagement is provided.
